(19) United States
(12) Patent Application Publication
(10) Pub. No.: US 2017/0156648 A1
(43) Pub. Date: Jun. 8, 2017

(54) METHOD AND APPARATUS FOR MEASURING HEMATOCRIT

(71) Applicant: GWANGJU INSTITUTE SCIENCE AND TECHNOLOGY, Buk-gu (KR)

(72) Inventors: Sung YANG, Buk-gu (KR); Myounggon KIM, Buk-gu (KR)

(21) Appl. No.: 15/434,675

(22) Filed: Feb. 16, 2017

Related U.S. Application Data
(62) Division of application No. 13/463,443, filed on May 3, 2012.

(30) Foreign Application Priority Data
Oct. 27, 2011 (KR) 10-2011-0110527

(51) Int. Cl. A61B 5/145; G01N 33/49; G01N 27/06

(52) U.S. Cl. CPC A61B 5/14535; G01N 27/06; G01N 33/49

(57) ABSTRACT

The present disclosure provides a method and apparatus for measuring hematocrit of blood. The method includes measuring resistance data of a target blood sample; calculating a hematocrit estimation parameter for measuring hematocrit of the target blood sample using the resistance data; and determining a hematocrit estimation value using the hematocrit estimation parameter. The method and apparatus for measuring hematocrit (HCT) provides convenience in measurement of hematocrit using electrical modeling.

### CROSS-REFERENCE TO RELATED APPLICATION

This present application is a Divisional Application of U.S. Ser. No. 13/473,443 filed May 3, 2012, which claims priority to Korean Patent Application No. 10-2011-0110527 filed on Oct. 27, 2011, and all the benefits accruing there from under 35 U.S.C. §119, the contents of which are incorporated by reference in their entirety.

### BACKGROUND

1. Technical Field

The present invention relates to a method and apparatus for measuring hematocrit. More particularly, the present invention relates to a method and apparatus for measuring hematocrit of blood using resistance data of the blood.

The present invention is derived from studies based on the Development Subject Research Agreement of the Gwangju Institute of Science and Technology [Title of the subject: System for monitoring patient in real time based on in-situ blood inspection, Case No. K02405] and World Class University Growth Business of the National Research Foundation of Korea [Title of the subject: Nanobio Material and Electronic Engineering, Case No N06820].

2. Description of the Related Art

Hematocrit (HCT) refers to the volume percent of red blood cells in blood. Generally, hematocrit is in the range of 37˜47% for a female and in the range of 45˜52% for a male. In a relatively simple method for measuring hematocrit, a blood sample is placed in a glass capillary tube and rotated at high speed such that red blood cells are separated from plasma to form a red blood cell layer in the tube. Then, the hematocrit is calculated by measuring the height of the red blood cell layer with respect to the entire height of the blood sample in the capillary tube.

Hematocrit is used as an indicator of blood conditions, such as blood circulation and anemia, and is measured for calibration of data regarding blood components such as blood glucose level and the like, which can be changed by the hematocrit.

Conventionally, centrifugation is applied to hematocrit measurement. For example, a blood sample is placed in a capillary tube and centrifuged, followed by measuring hematocrit of the blood sample using the ratio of the height of a red blood cell layer to 100% of the blood sample. This process takes a relatively long time and does not provide results in real time.

For a Coulter counter, the average volume percent and number of red blood cells are measured using electrical signals. The volume percent of red blood cells can be measured based on measurement data, but the Coulter counter is expensive, has a large volume and is difficult to carry.

### BRIEF SUMMARY

In electrical measurement of hematocrit, an error rate is likely to increase due to variation of plasma conditions such as osmotic pressure and electrical conductivity. This is because electric characteristics of blood depend not only on hematocrit (HCT) but also on an electrical state of plasma. Therefore, there is a need for development of a hematocrit measurement method which has a low error rate even in the case where the state of plasma varies.

In accordance with an aspect of the present invention, there is provided a method for measuring hematocrit of blood, which includes measuring resistance data of a target blood sample; calculating a hematocrit estimation parameter for measuring hematocrit of the target blood sample using the resistance data; and determining a hematocrit estimation value using the hematocrit estimation parameter.

The resistance data of the target blood sample may include resistances of plasma and cytoplasm of the target blood sample.

The hematocrit estimation parameter may be defined as a ratio of the resistance of the plasma to the sum of the resistances of the plasma and cytoplasm.

The hematocrit estimation value may be determined from the hematocrit estimation parameter using a predetermined linear function with regard to a relationship between the calculated hematocrit estimation parameter and the hematocrit.

The linear function may be determined by linear fitting of hematocrit values of individual test blood samples for a plurality of different test blood samples with hematocrit estimation parameters for the individual test blood samples.

The method may further include measuring an error rate by comparing the determined hematocrit estimation value with preset comparison data obtained using a Coulter counter or a micro centrifuge.

In accordance with another aspect of the present invention, there is provided an apparatus for measuring hematocrit of blood, which includes: a resistance data measurement unit which measures resistance data of a target blood sample including resistances of plasma and cytoplasm in the target blood sample; a parameter calculation unit which calculates a hematocrit estimation parameter for measuring hematocrit of the target blood sample using the resistance data; and a hematocrit measurement unit which determines a hematocrit estimation value using the hematocrit estimation parameter.

The hematocrit measurement unit may select the resistances of the plasma and cytoplasm from among the resistance data to calculate the hematocrit estimation parameter defined as a ratio of the resistance of the plasma to the sum of the resistances of the plasma and cytoplasm.

The hematocrit measurement unit may determine the hematocrit estimation value from the hematocrit estimation parameter using a predetermined linear function with regard to a relationship between the calculated hematocrit estimation parameter and the hematocrit.

The linear function may be determined linear fitting of hematocrit values of individual test blood samples for a plurality of different test blood samples with hematocrit estimation parameters for the individual test blood samples.

The apparatus may further include an error rate measurement unit which measures an error rate by comparing the determined hematocrit estimation value with preset comparison data obtained using a Coulter counter or a micro centrifuge.

In accordance with a further aspect of the present invention, there is provided a hematocrit measurement system, which includes a body formed of a non-conductive material; a blood receiving portion formed in the body and receiving blood; a plurality of electrodes having outer shells and connected to the blood receiving portion; and an apparatus for measuring hematocrit connected to the electrodes.

The apparatus for measuring hematocrit may include: a resistance data measurement unit which measures resistance data of a target blood sample including resistances of plasma and cytoplasm in the target blood sample; a parameter calculation unit which calculates a hematocrit estimation parameter for measuring the hematocrit using the resistance data; and a hematocrit measurement unit which determines a hematocrit estimation value using the hematocrit estimation parameter.

In accordance with yet another aspect of the present invention, there is provided a computer readable storage medium storing a computer program for implementing the method of measuring hematocrit according to any one of claims 1 to 6 in a computer.

### BRIEF DESCRIPTION OF THE DRAWINGS

The above and other aspects, features, and advantages of the present invention will become apparent from the detailed description of the following embodiments in conjunction with the accompanying drawings, in which:

FIG. 1 is a graph depicting a relationship between hematocrit (HCT) and resistance of blood as measured by a technique in the related art;

FIG. 2 is a diagram of blood data obtained through electrical modeling in the related art;

FIG. 3 is a graph depicting a relationship between resistance and reactance according to frequencies in electrical modeling of blood data in the related art;

FIGS. 4a to 4d are graphs depicting relationships between hematocrit and variables of blood data including Ri, Rp, CPE-C, and CPE-a;

FIG. 5 is a flowchart of a method of measuring hematocrit using a hematocrit estimation parameter according to one embodiment of the present invention;

FIG. 6 is a graph depicting a relationship between a hematocrit estimation parameter and hematocrit using a linear function according to one embodiment of the present invention;

FIG. 7 is a graph depicting a relative error rate calculated by comparing hematocrit estimation values calculated in the method according to the embodiment with preset reference data;

FIG. 8 is a block diagram of a hematocrit measurement apparatus according to one embodiment of the present invention; and

FIG. 9 is a block diagram of a hematocrit measurement system according to one embodiment of the present invention.

### DETAILED DESCRIPTION

Next, embodiments of the present invention will be described with reference to the accompanying drawings. Herein, description of details apparent to those skilled in the art will be omitted herein for clarity.

Among various electrical modeling methods, an electrical modeling method using impedance permits size reduction of peripheral devices for supporting a measurement apparatus in designing a measurement system and may reduce manufacturing cost, thereby providing good economic feasibility. In addition, since the electrical modeling method using impedance permits easy application of microfluidics as compared with other methods in the art, there is an increasing need for a method of electrically measuring hematocrit (HCT), that is, the volume percent of red blood cells.

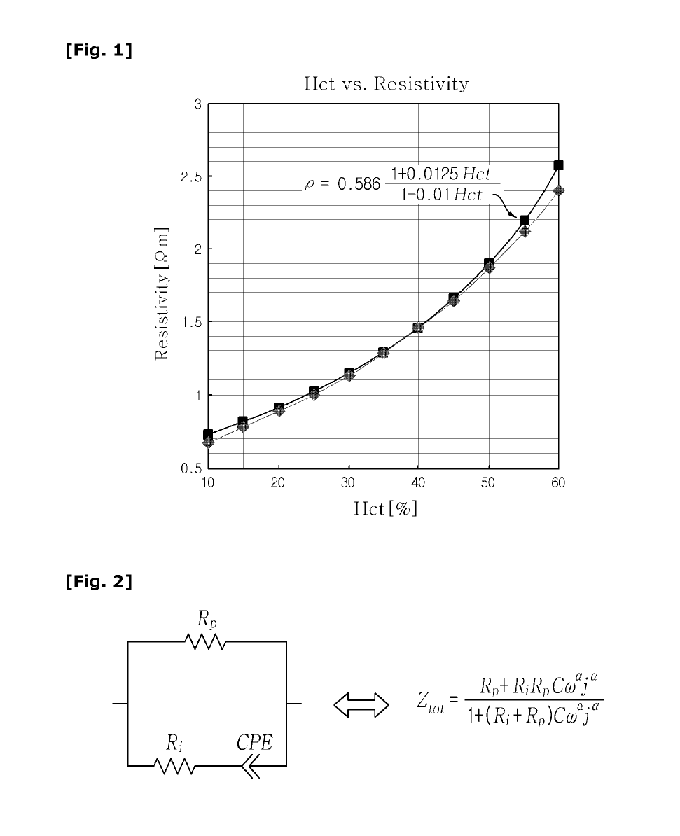

FIG. 1 is a graph depicting a relationship between resistance (p) and hematocrit of blood as measured by a method of electrically measuring hematocrit in the related art. In a conventional electric measurement method, hematocrit is electrically measured in real time using a microfluidic system. Such an electrical measurement method has a merit in that this method can be easily applied not only to the microfluidic system, but also to other existing systems.

σ_{whole blood}=fn(Hct, σₚₗₐₛₘₐ, πₚₗₐₛₘₐ) <Equation 1>

However, as expressed by Equation 1, in the conventional method of electrically measuring hematocrit (HCT), electric conductivity (σ_{whole blood}) of blood is affected by hematocrit and the electric conductivity (σₚₗₐₛₘₐ) and osmotic pressure (πₚₗₐₛₘₐ) of plasma in the blood. As a result, it is not easy to determine whether variation of electric parameters of the blood is caused by variation of the hematocrit or by variation of the plasma, and a measurement error increases in measurement of the hematocrit. In other words, there is a need for electrical parameters of blood which are mainly affected by the hematocrit instead of the electric conductivity (σₚₗₐₛₘₐ) and osmotic pressure (πₚₗₐₛₘₐ) of the plasma. Further, a conventional method of measuring hematocrit through electrical modeling based on electric permittivity (e) has an error rate of about 8.0% and thus there is a need for a hematocrit measurement method that has a low error rate.

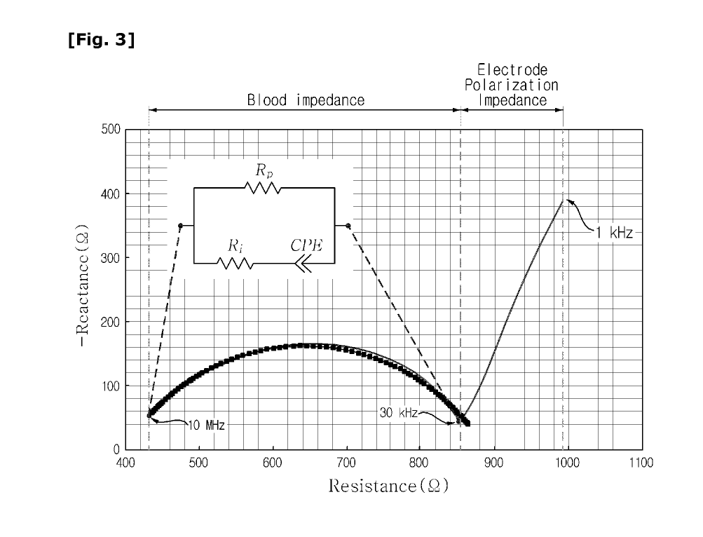

FIG. 2 is a diagram of blood data according to frequency variation, which are obtained through electrical modeling, in which Rₚ means resistance of plasma, Rᵢ means resistance of cytoplasm, and CPE (Constant Phase Element) represents frequency response characteristics of blood, which exhibits characteristics of a heterogeneous material composed of liquid and solid, and means dielectric dispersion in the frequency response of red blood cells and plasma, that is, capacitance which readily varies according to frequency.

$${Z}_{CPE} = \frac{1}{{C ( ω j )}^{α}} \qquad < Equation 2 >$$

Resistance (Z_{CPE}) of the CPE can be defined by Equation 2 and C (hereinafter, CPE-C) is a constant which denotes the intensity of CPE. (see Bao et al. (Bao, J. Z., Davis, C. C., Schmukler, R. E.), 1992. Biophys. J, 61(5), 1427-1434).

Further, “ω” represents the frequency variation and “j” is a complex number. “ω” and “j” are variables adjusted according to characteristics of frequencies. “a” represents the degree of dielectric dispersion by a plurality of cells, or surface roughness of the cells. The surface roughness tends to increase with decreasing value of a. (ω=2pf, 0<a<1)

The impedance of blood differs according to conditions. For example, hypotonic, isotonic, mannitol, and aqueous NaCl solutions provide different impedances of blood.

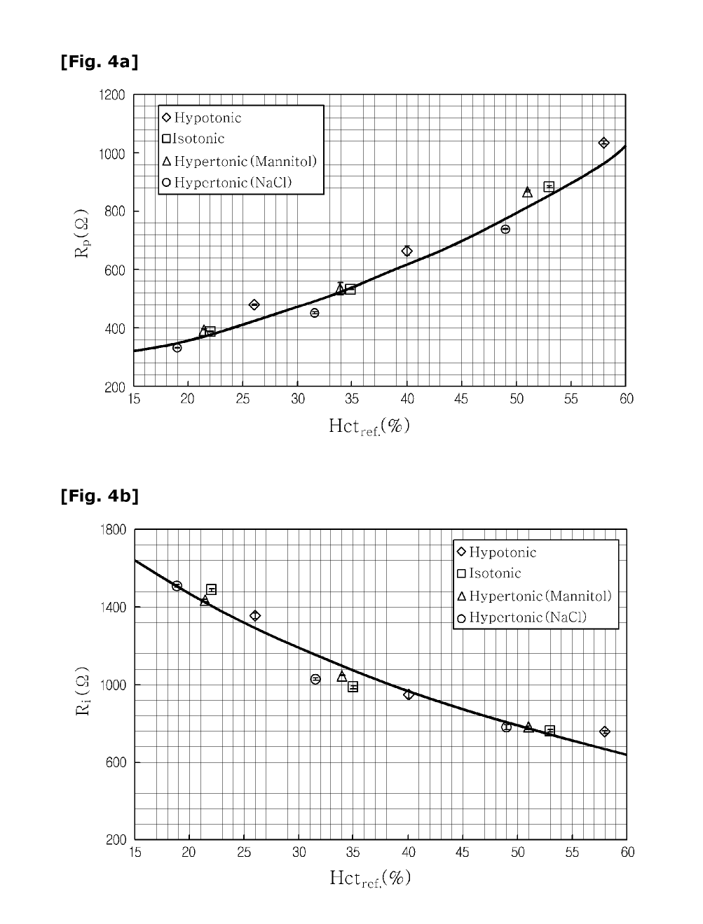

FIG. 3 is a graph depicting a relationship between resistance and reactance according to frequencies in electrical modeling of blood data in the related art, which shows impedance variation. Here, the resistance zone may be divided into a blood impedance zone and an electrode polarization impedance zone. A solid line indicates impedance data obtained through a resistance data measurement unit, and a dotted line indicates results obtained by fitting the blood impedance zone through electrical modeling. For example, while the frequency is varied from a low frequency of 1 kHz to a high frequency of 10 Mhz, the electrode impedance gradually decreases, and when the frequency is varied from 30 kHz to 10 Mhz, the resistance gradually decreases and the reactance increases according to the phase of the frequency.

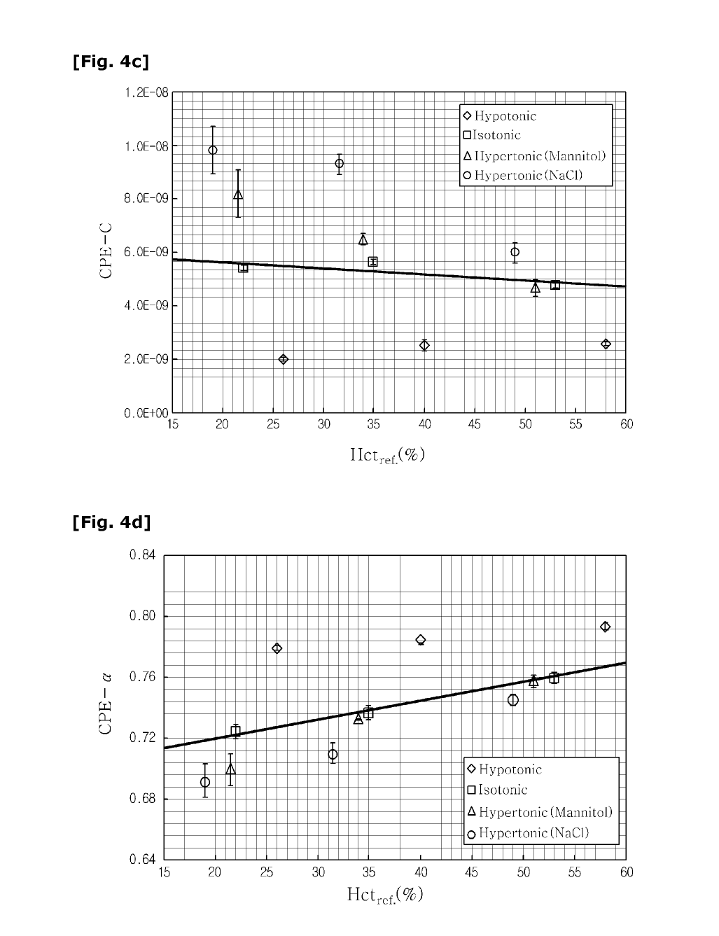

FIGS. 4a to FIG. 4d are graphs depicting variables for electrical modeling according to hematocrit concentration.

Here, Rₚ means the resistance of plasma, Rᵢ means the resistance of cytoplasm, CPE-C means the capacitor modification constant of CPE, that is, CPE-a (a of CPE). In these graphs, Rₚ tend to increase with increasing hematocrit and Rᵢ tend to decrease with increasing hematocrit.

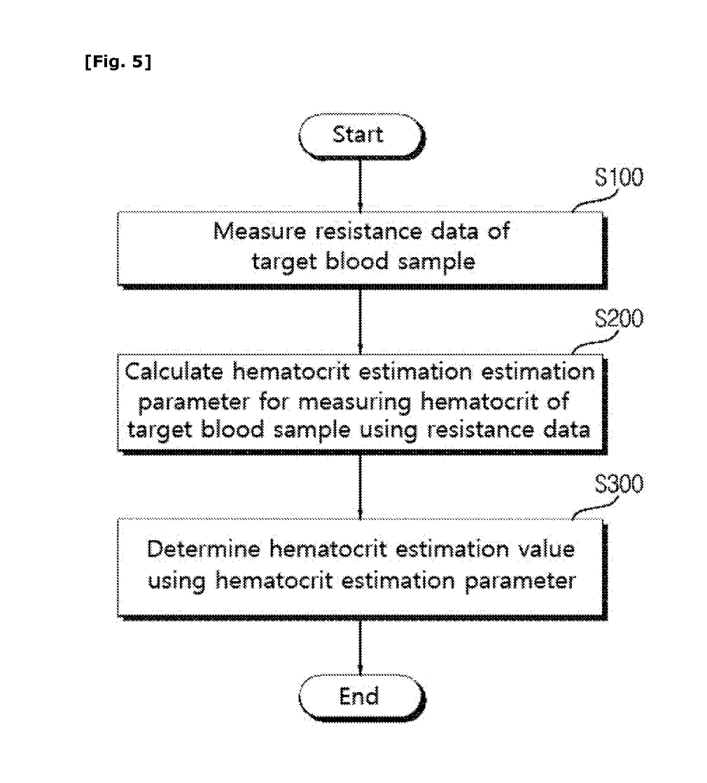

Next, a method of measuring hematocrit according to one embodiment of the invention will be described with reference to FIG. 5.

In S100, resistance data of a target blood sample prepared for measurement of hematocrit in blood are measured using an apparatus for measuring hematocrit. The resistance data may include the resistance and volume of plasma, and the resistance and volume of cytoplasm. In some embodiments, operation of measuring resistance data may include measuring the resistances of plasma and cytoplasm in the target blood sample through electrical modeling.

In S200, a hematocrit estimation parameter is calculated based on the resistance data of the target blood sample. In some embodiments, the resistance of plasma and the resistance of cytoplasm are selected from among the resistance data of the target blood sample to calculate the hematocrit estimation parameter, which is defined as the ratio of the resistance of the plasma to the sum of the resistances of the plasma and cytoplasm.

Namely, in a method of measuring hematocrit in a blood sample or an aqueous solution using two electrodes at low frequency, the blood and the aqueous solution may be modeled by resistance (R) expressed by the following Equation 3:

$$R - ρ \frac{L}{A} \qquad < Equation 3 >$$

where ρ is resistivity, L is the distance between two electrodes, and A is the contact area between the solution and the electrode. As the volume V of the blood sample or the solution decreases, R varies according to variation of A since ρ and L are constants. That is, as the volume V decreases, the contact area A between the solution and the electrode decreases and the resistance R increases due to decrease in contact area (A).

$$\frac{{V}_{R}}{{V}_{P} + {V}_{R}} \cdot 100 ( \% ) \approx \frac{\frac{1}{{R}_{i}}}{\frac{1}{{R}_{p}} + \frac{1}{{R}_{i}}} \cdot 100 ( \% ) = \frac{{R}_{P}}{{R}_{i} + {R}_{p}} \cdot 100 ( \% ) \qquad < Equation 4 >$$

Namely, as expressed by Equation 4, the resistance Rₚ of plasma is inversely proportional to an inverse number of the volume Vₚ of the plasma, and the resistance of Ri of cytoplasm inversely proportional to an inverse number of the volume V_{R} of red blood cells (RBCs).

Thus, according to the relationship between inverse numbers of the volume and the resistance, the hematocrit estimation parameter may be defined by Eauation 5.

$$\frac{{R}_{p}}{{R}_{i} + {R}_{p}} \qquad < Equation 5 >$$

In some embodiments, the hematocrit estimation parameter may be represented by the percent (%) of the resistance Rₚ of plasma to the sum of the resistance Rₚ of the plasma and the resistance Rᵢ of the cytoplasm.

Unlike the related art, the calculated hematocrit estimation parameter is modeling data, which do not include the electric conductivity and osmotic pressure of plasma. Thus, the calculated hematocrit estimation parameter is not changed due to variation of the electric conductivity or osmotic pressure of blood.

In S300, a hematocrit estimation value is determined using a linear function related to a relationship between the calculated hematocrit estimation parameter and the hematocrit.

In one embodiment, determination of the hematocrit estimation value using the linear function may include determining the linear function by linear fitting of predetermined hematocrit values of a plurality of different test blood samples with hematocrit estimation parameters for the individual test blood samples, and determining the hematocrit estimation value from the calculated hematocrit estimation parameter using the determined linear function.

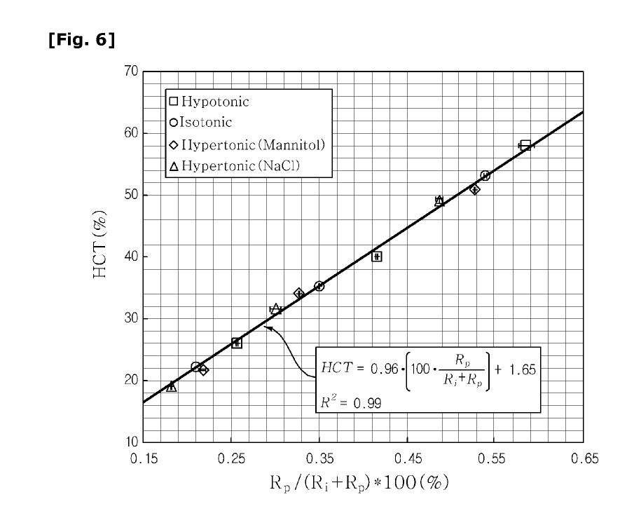

FIG. 6 shows a linear fitting relationship between the hematocrit values of individual test blood samples for a plurality of individual test blood samples and the hematocrit estimation parameters for the individual test blood samples, in which the linear function is obtained from data set to allow linear fitting of the hematocrit estimation parameters and the hematocrit values of the individual test samples by measuring a plurality of blood samples according to the electrical conductivity and osmotic pressure of plasma causing an increase of the error rate. In one embodiment, when the measured hematocrit estimation parameter is represented by Rₚ/(Rᵢ+Rₚ)*100, the hematocrit estimation values are linearly fitted with the hematocrit estimation parameters based on the data of the plurality of blood samples to predetermine corresponding data. Thus, assuming that a hematocrit estimation parameter is 0.35, the hematocrit estimation value is about 35%. In other words, referring to FIG. 6, it can be seen that all of the plurality of blood samples has a high linearity with respect to the measured hematocrit estimation parameters, and that the linear fitting results are represented by 0.96·[100·Rp/{Ri+Rp}]+1.65, R2=0.99.

P_{HCT}=a₁·[100·Rₚ/{Rᵢ+Rₚ}]+a₂ <Equation 6>

In Equation 6, al is a linear coefficient obtained by dividing a difference between maximum and minimum hematocrit estimation values (P_{HCT}) by a difference between maximum and minimum values of the hematocrit estimation parameter (100·Rₚ/{Rᵢ+Rₚ}), and is advantageously in the range from 0.90 to 0.99. In this embodiment, a1 is 0.96. a2 is a calibration coefficient for the hematocrit estimation value when the P_{HCT} has a minimum value, and is advantageously in the range from 1.0 to 4.5. In this embodiment, a2 is 1.65. If al and a2 are not within these ranges, the hematocrit estimation value has an error rate exceeding 4%, making it difficult to obtain desired effects. More specifically, the hematocrit may be measured through calibration using the linear fitting result within the range of the linear coefficients as described above.

In another embodiment, the method of measuring hematocrit may further include measuring an error rate by comparing the determined hematocrit value with preset comparison data.

In this embodiment, the preset comparison data may be set by a user and may include measurement results obtained using a Coulter counter or a micro centrifuge.

$$Error ( \% ) - \vert \frac{{HCT}_{ref} - {HCT}_{meas}}{{HCT}_{ref}} \vert \cdot 100 \qquad < Equation 7 >$$

Equation 7 represents a method of measuring an error rate by comparing the determined hematocrit estimation value HCTₘₑₐₛ with hematocrit HCT_{ref} measured by other existing devices. Here, the hematocrit estimation value (HCTₘₑₐₛ) means P_{HCT} measured by the method according to the embodiment of the invention. In other words, the method according to this embodiment may further include measuring the error range in order to ascertain precision of the measurement results obtained by the method according to the embodiment of the invention.

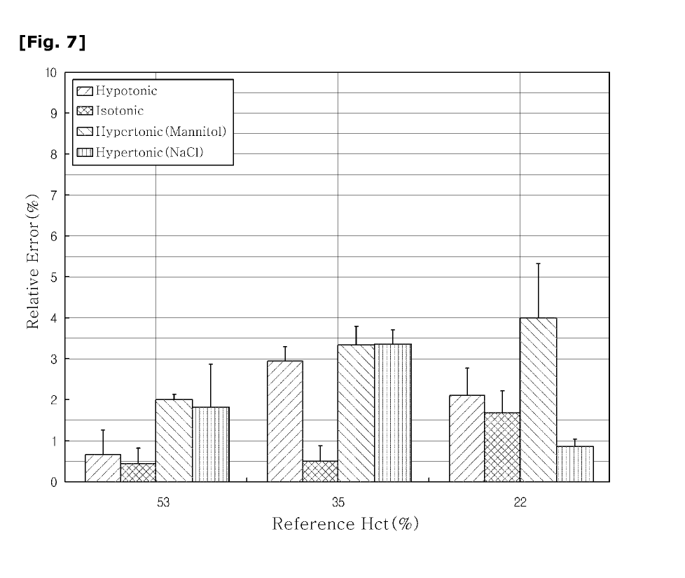

FIG. 7 is a graph depicting a relative error rate calculated by comparing hematocrit estimation values calculated by the method according to the embodiment with reference data. When a hematocrit value is 35% in reference data, an error range or a relative error rate of the hematocrit estimation value measured by the method according to the embodiment is less than 4% and thus it can be seen that the method according to the embodiment provides an effective measurement result.

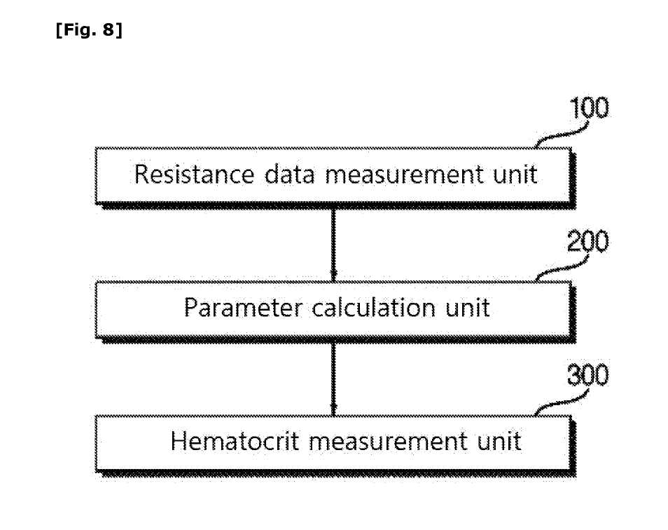

Next, an apparatus for measuring hematocrit according to one embodiment of the invention will be described with reference to FIG. 8.

The resistance data measurement unit 100 may measure resistance data that include resistances of plasma and cytoplasm in a target blood sample, hematocrit of which will be measured by the apparatus.

In some embodiments, blood data may include the resistance data, which will be used for measurement of the hematocrit and include the resistance and volume of plasma, and the resistance and volume of cytoplasm.

The parameter calculation unit 200 may calculate a hematocrit estimation parameter based on the resistance data measured by the resistance data measurement unit 100. In some embodiments, the parameter calculation unit 200 may calculate the hematocrit estimation parameter by selecting the resistances of the plasma and cytoplasm from among the resistance data, in which the hematocrit estimation parameter is defined by the ratio of the resistance of the plasma to the sum of the resistances of the plasma and cytoplasm.

The hematocrit measurement unit 300 may determine a hematocrit estimation value using a predetermined linear function relating to a relationship between the calculated hematocrit estimation parameter and the hematocrit. In some embodiments, determination of the hematocrit estimation value using the linear function may include determining the linear function by linear fitting of predetermined hematocrit values of a plurality of different test blood samples with hematocrit estimation parameters for the individual test blood samples, and determining the hematocrit estimation value of the target blood sample from the calculated hematocrit estimation parameter using the determined linear function.

The apparatus may further include an error measurement unit which measures an error rate by comparing the determined hematocrit value with preset comparison data. The preset comparison data may include data, which are set by measuring a plurality of data samples according to the electrical conductivity and osmotic pressure of plasma causing an increase of the error rate.

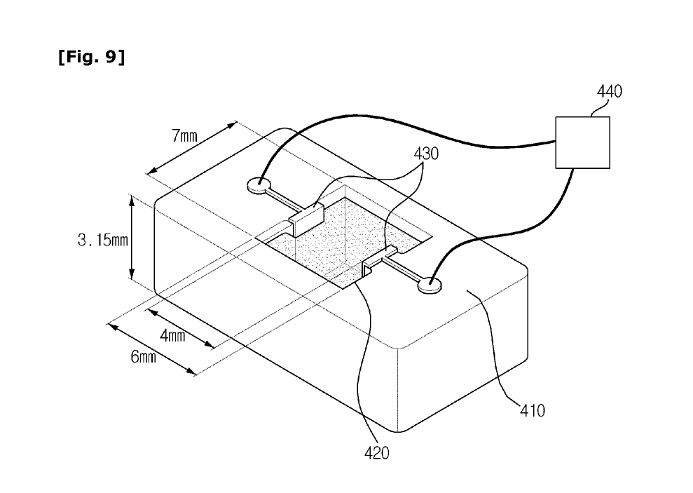

Next, a hematocrit measurement system according to one embodiment of the invention will be described with reference to FIG. 9. Here, the apparatus for measuring hematocrit has the same configuration as descried above, and a detailed description thereof will be omitted herein.

The body 410 may be made of a non-conductive material. In some embodiments, the body 410 may be made of a plastic material, which is a non-conductive material. The body has a height so as to define the blood receiving portion therein. That is, since red blood cells are heavier than plasma, the body has a height ranging from 2.5 to 4 mm in order to prevent a measurement error caused by the red blood cells sinking in the blood receiving portion. Advantageously, the body has a height of 3.15 mm.

The blood receiving portion 420 is defined within the body to receive blood supplied from the outside. Namely, the blood receiving portion may store a target blood sample supplied via a device such as a pipette or the like. A plurality of electrodes is attached to opposite sides of the blood receiving portion 420 to face each other in a longitudinal direction of the body and each of the electrodes has a predetermined thickness and width. To guarantee accurate measurement of hematocrit, the electrodes attached to the opposite sides may be separated a certain distance from each other. To this end, the opposite sides of the blood receiving portion 420 are advantageously separated a distance ranging from 5 mm to 7 mm from each other in the longitudinal direction of the body. In this embodiment, the opposite sides of the blood receiving portion 420 are separated a distance of 6 mm from each other in the longitudinal direction of the body. Further, for accurate measurement of the resistance through the electrodes, the blood receiving portion 420 has other opposite sides which are separated from each other in a transverse direction of the body so as not to be excessively separated from the electrodes. Advantageously, the opposite sides of the blood receiving portion 420 are separated a distance of 6 mm to 8 mm from each other in the transverse direction. In this embodiment, the opposite sides of the blood receiving portion 420 are separated a distance of 7 mm from each other in the transverse direction.

The plural electrodes 430 are attached to opposite sides of the blood receiving portion in the longitudinal direction of the body and have outer shells to prevent failure or contamination caused by blood. In measurement of the resistance through the plural electrodes, there can be a possibility of an error due to insufficient flow of electric current in blood if the electrodes are separated an appropriate distance apart. Thus, the electrodes are separated by a distance ranging from 4 mm to 6 mm. In this embodiment, the electrodes are separated by a distance of 5 mm.

The hematocrit measurement apparatus 440 is connected to the electrodes, and includes a resistance data measurement unit, a parameter calculation unit and a hematocrit measurement unit. In some embodiments of the invention, the apparatus for measuring hematocrit may further include an impedance tester (4294A, Agilent Technologies Inc., Calif., U.S.A.). The hematocrit measurement apparatus 440 has the same configuration as described above and further elaboration thereof will be omitted herein.

The resistance data measurement unit may measure resistance data that include resistances of plasma and cytoplasm in a target blood sample, hematocrit of which will be measured by the apparatus.

The parameter calculation unit may calculate a hematocrit estimation parameter based on the resistance data measured by selecting the resistances of the plasma and cytoplasm from among the resistance data, in which the hematocrit estimation parameter is defined by the ratio of the resistance of the plasma to the sum of the resistances of the plasma and cytoplasm.

The hematocrit measurement unit may determine a hematocrit estimation value using a predetermined linear function relating to a relationship between the calculated hematocrit estimation parameter and the hematocrit.

The above and other embodiments of the present invention may be implemented by a computer program. Code and code segments of the computer program may be easily devised by computer programmers in the art. Further, the computer program may be stored in computer readable storage media and may be read and implemented by a computer to implement the embodiments. The computer readable storage media may include magnetic storage media, optical recording media, and carrier wave media.

As such, the method and apparatus for measuring hematocrit (HCT) according to embodiments of the present invention provide convenience in measurement of hematocrit through electrical modeling.

Although some embodiments have been described herein, it should be understood by those skilled in the art that these embodiments are given by way of illustration only, and that various modifications, variations, and alterations can be made without departing from the spirit and scope of the invention. Therefore, the embodiments and the accompanying drawings should not be construed to limit the technical spirit of the present invention, but should be construed to illustrate the spirit of the present invention. The scope of the invention should be interpreted according to the following appended claims to cover all modifications or variations induced from the appended claims and equivalents thereof.

What is claimed is:

1. A hematocrit measurement system comprising:
a body formed of a non-conductive material;
a blood receiving portion formed in the body to receive blood;
a plurality of electrodes having outer shells and connected to the blood receiving portion; and
an apparatus for measuring hematocrit connected to the electrodes.

2. The hematocrit measurement system according to claim 12, wherein the apparatus comprises:
a resistance data measurement unit which measures resistance data of a target blood sample including resistances of plasma and cytoplasm in the target blood sample;
a parameter calculation unit which calculates a hematocrit estimation parameter for measuring hematocrit of the target blood sample using the resistance data; and
a hematocrit measurement unit which determines a hematocrit estimation value using the hematocrit estimation parameter.

3. The hematocrit measurement system according to claim 13, wherein the hematocrit measurement unit selects the resistances of the plasma and cytoplasm from among the resistance data to calculate the hematocrit estimation parameter defined as a ratio of the resistance of the plasma to the sum of the resistances of the plasma and cytoplasm, and the hematocrit measurement unit determines the hematocrit estimation value from the hematocrit estimation parameter using a predetermined linear function with regard to a relationship between the calculated hematocrit estimation parameter and the hematocrit.

4. The hematocrit measurement system according to claim 14, wherein the linear function is determined by linear fitting of predetermined hematocrit values of a plurality of different test blood samples with hematocrit estimation parameters for the individual test blood samples.
